(19) United States
(12) Patent Application Publication
(10) Pub. No.: US 2015/0164170 A1
(43) Pub. Date: Jun. 18, 2015

(54) Crash Helmet

(71) Applicant: Changlong Zeng, Shenzhen (CN)

(72) Inventor: Changlong Zeng, Shenzhen (CN)

(21) Appl. No.: 14/132,315

(22) Filed: Dec. 18, 2013

(51) Int. Cl. A42B 3/04; A42B 3/06; A61F 9/02

(52) U.S. Cl. CPC A42B 3/0453; A61F 9/02; A42B 3/06

(57) ABSTRACT

A crash helmet includes a body, a remote controlled emitter, a turning indicator light in a shape of an arrow and located at two sides of the body, a flasher located at a middle portion of a back of the crash helmet, a remote controlled receptor electrically connected with the turning indicator light and the flasher, and a power module located at the body of the crash helmet and is configured for providing power to the turning indicator light, the flasher, and the remote controlled receptor. When the rider wears the crash helmet of the disclosure, it is clear for a person walking behind the rider or a driver behind the rider to understand the moving direction of the rider, which is safe and avoids traffic accidents. The flasher is capable of flashing for a long time, which enhance safe for the rider in the nighttime.

### BACKGROUND

1. Technical Field

The present disclosure generally relates to crash helmets.

2. Description of Related Art

A crash helmet is generally configured for protecting a head of a rider. When the rider is crashed, the crash helmet protects the head of the rider from hurt and even saves a life of the rider. So that the crash helmet is an indispensable instrument in traffic fields. Some crash helmets with special functions generally include earphones, microphone, and interfaces for mounting cameras and flashlights. Although the crash helmets generally have the function of indicating turning of bicycles, when the rider indicates the turning of the bicycle, the rider has to lose hold of the handle of the bicycle to indicate the turning of the bicycle by the hand, or contact a switch on the crash helmet by the hand to turn on the turning light. This way of indicating turning of the bicycles is effective, but it is prone to be a safety hidden danger to the rider, due to the departure of the hand of the rider from the handle of the bicycle.

Therefore, a need exists in the industry to overcome the described problems.

### SUMMARY

The disclosure is to solve a problem that it is prone to be a safety hidden danger to a rider, due to the departure of the hand of the rider from a handle of a bicycle when the rider turns on the turning light on the crash helmet. The target of the disclosure is to provide a crash helmet for a bicycle which is safe for the rider and prevents traffic accidents effectively.

In order to solve the problem described above, the disclosure offers a technical proposal as following.

A crash helmet includes a body, a remote controlled emitter, a turning indicator light, a flasher, a remote controlled receptor, and a power module. The turning indicator light is in a shape of an arrow and located at two sides of the body. The flasher is located at a middle portion of a back of the body of the crash helmet. The remote controlled receptor is located at the middle portion of the back of the body of the crash helmet, and is electrically connected with the turning indicator light and the flasher. The power module is located at the body of the crash helmet and is configured for providing power to the turning indicator light, the flasher, and the remote controlled receptor.

The flasher is a clearance light in a shape of a triangle.

The remote controlled emitter is mounted on a handle of a bicycle.

The crash helmet further includes a belt located at the body of the crash helmet, and configured to to prevent the crash helmet dropping from a head of the rider.

The body defines a receiving slot receiving the remote controlled emitter.

The power module is a rechargeable battery or a solar battery.

The crash helmet further includes a buzzer electrically connected to the turning indicator light. When the flasher flashes, the crash helmet generates sound like “didi” to inform the rider that the indicator light on the crash helmet works normally.

The crash helmet further includes a protective goggle to protect the rider completely.

When the rider wears the crash helmet of the disclosure, it is clear for a person walking behind the rider or a driver behind the riders to understand the moving direction of the rider, which is safe and can avoids traffic accidents effectively. The flasher located at the middle portion of the back of the crash helmet is capable of flashing for a long time, which enhances safe for the rider in the nighttime. Because the flasher is the clearance light in the shape of the triangle, when the rider stops the bicycle to have a break or to repair the bicycle, the crash helmet can be located at a place far away from the bicycle about serial meters, and the crash helmet is configured as a triangle-shaped alarming frame. The remote controlled emitter is mounted on the handle of the bicycle, so that the rider can control the turning indicator light without departure of the hand from the handle of the bicycle, which ensures safe of the rider. In addition, when the remote controlled emitter is unused, the remote controlled emitter is detached from the handle of the bicycle and is positioned in the receiving slot of the crash helmet, which reduces a risk of loss of the remote controlled emitter.

In order to further understand feature and content technology of the disclosure, please refer to the detail description and drawing of the disclosure. However, the drawing is only for reference and description, does not be a limitation for the disclosure.

### BRIEF DESCRIPTION OF THE DRAWINGS

Many aspects of the present embodiments can be better understood with reference to the following drawings. The components in the drawings are not necessarily drawn to scale, the emphasis instead being placed upon clearly illustrating the principles of the present embodiments. Moreover, in the drawings, all the views are schematic, and like reference numerals designate corresponding parts throughout the several views.

FIG. 1 is an assembled view of a crash helmet of an exemplary embodiment of the disclosure.

### DETAILED DESCRIPTION

The disclosure is illustrated by way of example and not by way of limitation in the figures of the accompanying drawings, in which like reference numerals indicate similar elements. It should be noted that references to “an” or “one” embodiment in this disclosure are not necessarily to the same embodiment, and such references can mean “at least one” embodiment.

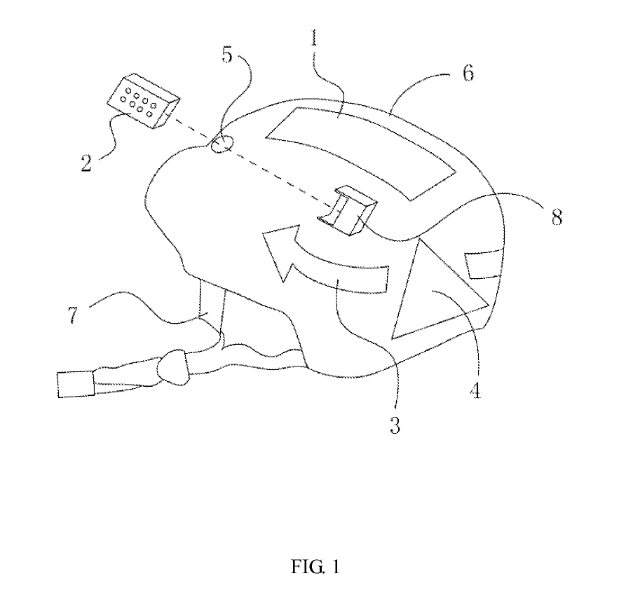

With reference to FIG. 1, a crash helmet includes a body 1, a remote controlled emitter 2, a turning indicator light 3, a flasher 4, a remote controlled receptor 5, and a power module 6. The turning indicator light 3 is in a shape of an arrow and located at two sides of the body 1. The flasher 4 is located at a middle portion of a back of the body 1 of the crash helmet. The remote controlled receptor 5 is located at the middle portion of the back of the body 1 of the crash helmet, and is electrically connected with the turning indicator light 3 and the flasher 4. The power module 6 is located at the body 1 of the crash helmet and is configured for providing power to the turning indicator light 3, the flasher 4, and the remote controlled receptor 5.

The flasher 4 is a clearance light in a shape of a triangle.

The remote controlled emitter 2 is mounted at a handle of a bicycle.

The crash helmet further includes a belt 7 located at the body 1 of the crash helmet to prevent the crash helmet dropping from a head of the rider.

The body 1 defines a receiving slot 8 receiving the remote controlled emitter 2.

The power module 6 is a rechargeable battery or a solar battery.

The crash helmet further includes a buzzer electrically connected to the turning indicator light 3. When the flasher 4 flashes, the crash helmet generates sound like “di di” to inform the rider that the turning indicator light 3 on the crash helmet works normally

The crash helmet further includes a protective goggle to protect the rider completely.

When the rider wears the crash helmet of the disclosure, it is clear for a person walking behind the rider or a driver behind the rider to understand the moving direction of the rider, which is safe and avoids traffic accidents effectively.

The flasher 4 located at the middle portion of the back of the crash helmet is capable of flashing for a long time, which enhances safe for the rider in the nighttime.

Because the flasher 4 is the clearance light in the shape of the triangle, when the rider stops the bicycle to have a break or to repair the bicycle, the crash helmet can be located at a place far away from the bicycle about serial meters, and the crash helmet is configured as a triangle-shaped alarming frame.

The remote controlled emitter 2 is mounted on the handle of the bicycle, so that the rider can control the turning indicator light 3 without departure of the hand from the handle of the bicycle, which ensures safe of the rider.

In addition, when the remote controlled emitter 2 is unused, the remote controlled emitter 2 is detached from the handle of the bicycle and is positioned in the receiving slot 8 of the crash helmet, which reduces a risk of loss of the remote controlled emitter 2.

Although the features and elements of the present disclosure are described as embodiments in particular combinations, each feature or element can be used alone or in other various combinations within the principles of the present disclosure to the full extent indicated by the broad general meaning of the terms in which the appended claims are expressed.

What is claimed is:

1. A crash helmet, comprising:
a body;
a remote controlled emitter;
a turning indicator light in a shape of an arrow and located at two sides of the body;
a flasher located at a middle portion of a back of the body of the crash helmet;
a remote controlled receptor located at the middle portion of the back of the body of the crash helmet, and electrically connected with the turning indicator light and the flasher; and
a power module located at the body of the crash helmet and configured for providing power to any one of the turning indicator light, the flasher, and the remote controlled receptor.

2. The crash helmet of claim 1, wherein the flasher is a clearance light in a shape of a triangle.

3. The crash helmet of claim 1, wherein the remote controlled emitter is located at a handle of a bicycle.

4. The crash helmet of claim 1, further comprising a belt located at the body of the crash helmet.

5. The crash helmet of claim 1, wherein the body of the crash helmet defines a receiving slot receiving the remote controlled emitter.

6. The crash helmet of claim 1, wherein the power module is selected from one of a rechargeable battery and a solar battery.

7. The crash helmet of claim 1, further comprising a buzzer electrically connected to the turning indicator light.

8. The crash helmet of claim 1, further comprising a protective goggle.
